(19) United States
(12) Patent Application Publication
(10) Pub. No.: US 2025/0210143 A1
(43) Pub. Date: Jun. 26, 2025

(54) qPCR Curve Detection

(71) Applicant: LIFE TECHNOLOGIES CORPORATION, Carlsbad, CA (US)

(72) Inventors: Yong Chu, Castro Valley, CA (US); Deepankar Chanda, San Francisco, CA (US); Nivedita Sumi Majumdar, San Bruno, CA (US); Wallace George, Oakland, CA (US); Ming Jiang, San Ramon, CA (US); Anurag Gautam, Tracy, CA (US); Nicolas Zun Yu Wong, San Francisco, CA (US); Yun Zhu, San Diego, CA (US); Avi Moshe Shapiro, Berkeley, CA (US); Vadim Mozhayskiy, San Diego, CA (US); Jasmine Patil, Mountain View, CA (US); Dhvani Pratik Patel, San Jose, CA (US)

(21) Appl. No.: 18/846,809

(22) PCT Filed: Mar. 15, 2023
(86) PCT No.: PCT/US2023/015326

§ 371 Date: Sep. 13, 2024

Related U.S. Application Data
(60) Provisional application No. 63/320,213, filed on Mar. 15, 2022.

(51) Int. Cl. G16B 40/10; G16B 40/20

(52) U.S. Cl. CPC G16B 40/10; G16B 40/20

(57) ABSTRACT

Embodiments of the invention are disclosed that provide improved computer systems, computerized methods, and computer program products for generating and evaluating automated predictions regarding whether a particular amplification curve from a qPCR assay indicates presence of a target molecule in a sample. In some embodiments, predictions are generated using deep learning networks. In some embodiments, curve-quality predictions are generated and used to assess whether an amplification prediction can be reliably made from a particular amplification curve or whether the curve reflects an anomaly in the qPCR assay. In various embodiments, prediction confidence data is also generated and used, along with prediction data, in an electronic user interface to improve qPCR measurement.

### CROSS-REFERENCE TO RELATED APPLICATIONS

This application claims the benefit of U.S. Provisional Application 63/320,213 filed on Mar. 15, 2022. To the extent permitted in applicable jurisdictions, the entire contents of that application are incorporated herein by reference.

### BACKGROUND

This disclosure relates generally to technology for determining whether a target is present in a sample.

An amplification curve obtained from a real-time (also known as quantitative) polymerase chain reaction (qPCR) experiment can be used to determine whether a target is present in a biological sample (e.g., a blood or food sample). In a typical qPCR experiment, fluorescence of a sample is measured after each thermal cycle of the experiment. The set of fluorescence values versus cycle number associated with a particular assay on a sample forms an amplification curve. Traditionally, an algorithm analyzes and/or a human reviews the amplification curve and, based on visual or other analysis of the curve's characteristics, determines whether the relevant sample amplified, which in turn indicates whether the target molecule was present within the sample. A typical algorithmic technique to determine that the relevant sample has amplified involves determining whether the associated amplification curve has crossed a threshold value that is either fixed or is calculated based on the characteristics of the amplification curve. If the threshold is crossed, the curve is determined to represent amplification; if the threshold is not crossed, the curve is determined to represent non-amplification.

### SUMMARY

Automated determination of amplification is important for increasing throughput of sample analysis, which in turn can both advance scientific research and improve provision of time-sensitive, clinically important information. Existing methods of automatically determining amplification have relied on combinations of techniques and parameters to improve accuracy. Machine learning techniques, including deep learning networks such as artificial neural networks, can help improve accuracy. Improved machine learning techniques are needed to further improve accuracy and reduce the instances in which human review is required. At the same time, it is important to effectively and optimally determine when human review is needed, and the criteria for triggering human review of amplification curves that have been evaluated by a computer system can vary by application context. Improving the overall call-rate accuracy of an amplification-calling machine learning system does not necessarily fully address the problem of knowing which individual amplification curve automated call results are at most risk of being wrong and therefore require human review.

Embodiments of the invention address aspects of this “which curve” problem by using an improved amplification calling machine learning system (in some embodiments, an improved artificial neural network) in parallel with a machine learning system (e.g., a deep learning network such as an artificial neural network) for evaluating the quality of an amplification curve. This can help identify potential problem curves. In some embodiments, alternative amplification calling algorithms are used to help determine which curves called by a machine-learning (e.g., neural network) amplification-calling system should be evaluated further for potential curve-quality issues that might require invalidating results.

In some embodiments, evidential learning techniques are used to extract call confidence data from both an amplification calling neural network and a curve-quality calling neural network. The confidence data can be used to further evaluate when a machine-generated amplification call should be reviewed by a human.

Some embodiments also provide improved amplification-calling and curve-quality calling networks based on particular types, variations, and arrangements of neural network elements, pre-processing and processing techniques, and/or engineered features used in processing the amplification curves from a qPCR assay. Further details of these embodiments are more fully-disclosed herein.

### BRIEF DESCRIPTION OF THE DRAWINGS

FIG. 1 illustrates a PCR test and analysis system in accordance with an embodiment of the present disclosure.

FIG. 2 is a high-level block architecture diagram of a computer system in accordance with an embodiment of the disclosure.

FIG. 3 illustrates pre-processing implemented in one embodiment of the present disclosure.

FIG. 4 is a high-level block diagram of an architecture of an amp-calling artificial neural network according to one embodiment of the disclosure.

FIG. 5 is a block diagram illustrating the structure of FIG. 4 in further detail in accordance with one embodiment of the present disclosure.

FIG. 6 is a block diagram of an architecture of a curve-quality calling artificial neural network according to one embodiment of the disclosure.

FIG. 7 illustrates computer processing to implement amp and curve-quality call evaluation in accordance with an embodiment of the disclosure.

FIG. 8 is a high-level block diagram of an architecture of an amp-calling artificial neural network according to one embodiment of the disclosure.

FIG. 9 is a block diagram illustrating the structure of FIG. 8 in further detail in accordance with one embodiment of the present disclosure.

FIG. 10 is a block diagram of an architecture of a curve-quality calling artificial neural network according to one embodiment of the disclosure.

FIG. 11 illustrates computer processing to implement confidence processing in accordance with an embodiment of the disclosure.

FIG. 12 illustrates an exemplary computer system configurable by a computer program product to implement embodiments of the present disclosure.

While the invention is described with reference to the above drawings, the drawings are intended to be illustrative, and other embodiments are consistent with the spirit, and within the scope, of the invention.

### DETAILED DESCRIPTION

The various embodiments now will be described more fully hereinafter with reference to the accompanying drawings, which form a part hereof, and which show, by way of illustration, specific examples of practicing the embodiments. This specification may, however, be embodied in many different forms and should not be construed as limited to the embodiments set forth herein; rather, these embodiments are provided so that this specification will be thorough and complete, and will fully convey the scope of the invention to those skilled in the art. Among other things, this specification may be embodied as methods or devices. Accordingly, any of the various embodiments herein may take the form of an entirely hardware embodiment, an entirely software embodiment or an embodiment combining software and hardware aspects. The following specification is, therefore, not to be taken in a limiting sense.

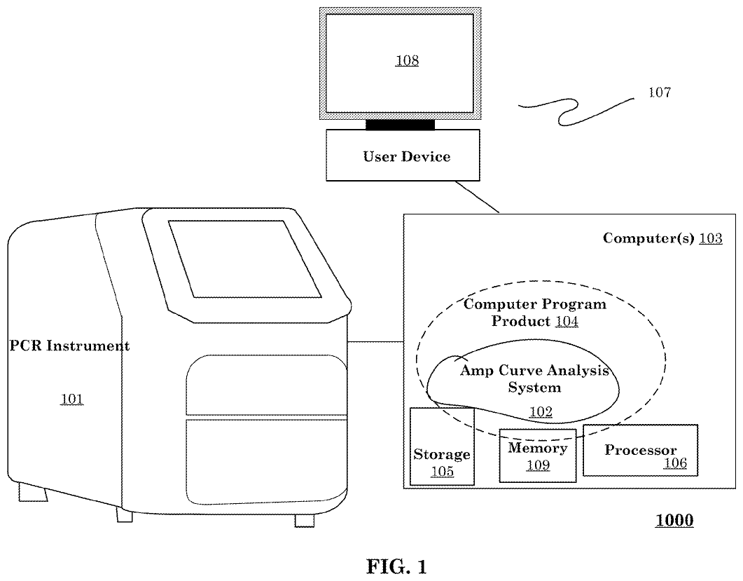

FIG. 1 illustrates System 1000 in accordance with an exemplary embodiment of the present disclosure. System 1000 comprises polymerase chain reaction (“PCR”) Instrument 101, one or more computers 103, and user device 107.

Instructions for implementing amplification (“amp”) curve analysis system 102 reside in computer program product 104 which is stored in storage 105 and those instructions are executable by processor 106. When processor 106 is executing the instructions of computer program product 104, the instructions, or a portion thereof, are typically loaded into working memory 109 from which the instructions are readily accessed by processor 106. In the illustrated embodiment, computer program product 104 is stored in storage 105 or another non-transitory computer readable medium (which may include being distributed across media on different devices and different locations). In alternative embodiments, the storage medium is transitory.

In one embodiment, processor 106 in fact comprises multiple processors which may comprise additional working memories (additional processors and memories not individually illustrated) including a graphics processing unit (GPU) comprising at least thousands of arithmetic logic units supporting parallel computations on a large scale. GPUs are often utilized in deep learning applications because they can perform the relevant processing tasks more efficiently than can typical general-purpose processors (CPUs). Other embodiments comprise one or more specialized processing units comprising systolic arrays and/or other hardware arrangements that support efficient parallel processing. In some embodiments, such specialized hardware works in conjunction with a CPU and/or GPU to carry out the various processing described herein. In some embodiments, such specialized hardware comprises application specific integrated circuits and the like (which may refer to a portion of an integrated circuit that is application-specific), field programmable gate arrays and the like, or combinations thereof. In some embodiments, however, a processor such as processor 106 may be implemented as one or more general purpose processors (preferably having multiple cores) without necessarily departing from the spirit and scope of the present invention.

User device 107 incudes a display 108 for displaying results of processing carried out by amp curve analysis system 102. In alternative embodiments, amp curve analysis system 102, or a portion thereof, may be stored in storage devices and executed by one or more processors residing on PCR instrument 101 and/or user device 107. Such alternatives do not depart from the scope of the invention.

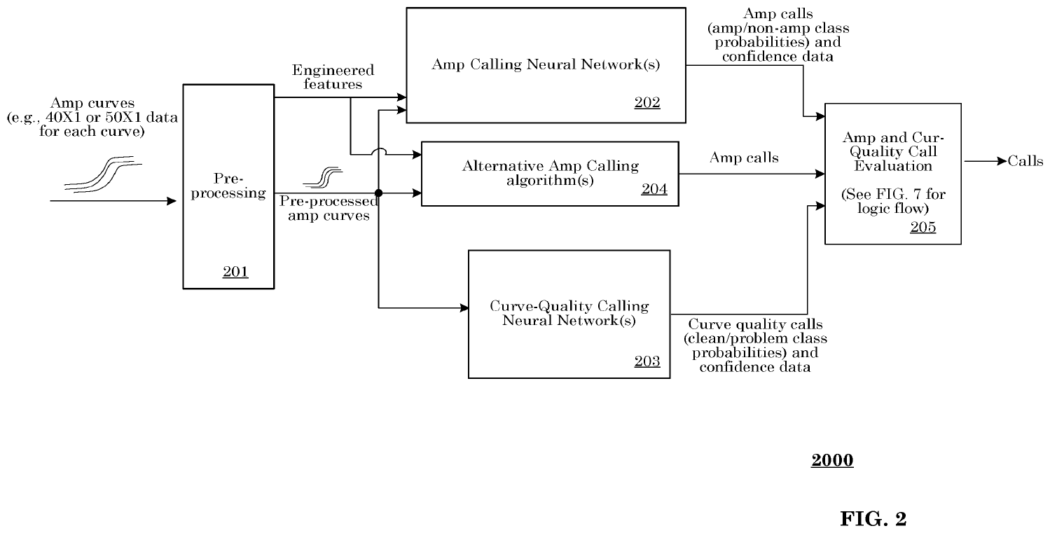

FIG. 2 is a high-level block architecture diagram of a computer system 2000 in accordance with an embodiment of the disclosure. Computer system 2000 comprises a pre-processing block 201, one or more amplification calling neural networks 202, one or more curve-quality calling neural networks 203, one or more alternative amplification calling algorithms 204, and amplification and quality call evaluation block 205.

Pre-processing block 201 is typically configured to receive one or more amplification curves corresponding to amplification data obtained from a 40-cycle or a 50-cycle qPCR assay. In alternative embodiments, amplification curves resulting from PCR experiments with other amounts of cycles can be processed by the illustrated embodiment using various techniques. The collection of discrete data points, for example, 40 fluorescence values for a 40-cycle PCR experiment (or 50 for a 50-cycle experiment) is referred to as a “curve” herein, even though it is not continuous data. At the same time, a best-fit continuous curve may or may not be fit to the data for easier visual display and/or analysis purposes. In any event, the term amplification “curve” herein will generally be used to refer to the set of discrete amplification data obtained and analyzed for an assay of a biological sample, unless another meaning is implied from the context.

Pre-processing block 201 processes amplification curves to generate pre-processed curves and engineered features. The term “engineered” features refers to various features obtained from pre-determined computations on an amplification curve and is distinguished from “learned” features that result from a submitting amplification curves to a neural network or network portion (e.g., convolutional layers of a neural network). In some embodiments of the disclosure, engineered features can be obtained during pre-processing and submitted to neural networks along with the pre-processed amplification curves and can enhance learning speed and/or accuracy relative to submitting only the pre-processed curves themselves. In some embodiments of the disclosure, engineered features include one or more of curve derivatives (e.g., first, second, and/or other higher order derivatives), time series features, and other features, such as, for example, features from various PCR analysis algorithms including PCRedux features and/or Cycle Relative Threshold features, as further defined and explained elsewhere herein.

In addition to processing received amplification curves to obtain engineered features, pre-processing block 201 also processes the amplification curves to remove baseline signals, elongate or shorten the curves to be a uniform length (e.g., 40-cycle or 50-cycle) and normalizes the curves. These operations are used to generate pre-processed amplification curves output by block 201.

Amplification (“amp”) calling neural network(s) 202, alternative amp calling algorithm(s) 204, and curve-quality calling neural network(s) 203 receive the pre-processed amplification curves. In the illustrated embodiment, amp calling neural network(s) 202 receive the engineered features from pre-processing block 201 and alternative amp-calling algorithms 204 receive a portion of the engineered features relevant for executing certain alternative amp calling algorithms. In alternative embodiments, curve-quality calling neural networks 203 also receive engineered features from a pre-processing block such as pre-processing block 201.

Amplification and curve-quality calling evaluation block 205 receives amplification call data from amp-calling neural network(s) 202. In the illustrated embodiment, the amplification call data includes class probabilities for at least an amplified class and a non-amplified class and further includes confidence data generated from evidential learning techniques. However, in alternative embodiments, confidence data is not generated and used. Block 205 also receives amplification call data from alternative amp-calling algorithm(s) 204. Block 205 receives curve-quality call data from curve-quality calling neural network(s) 203. In the illustrated embodiment, the curve-quality call data includes class probabilities for at least a “clean” curve class and an anomalous (“problem”) curve class and further includes confidence data generated from evidential learning techniques. However, in alternative embodiments, confidence data is not generated and used.

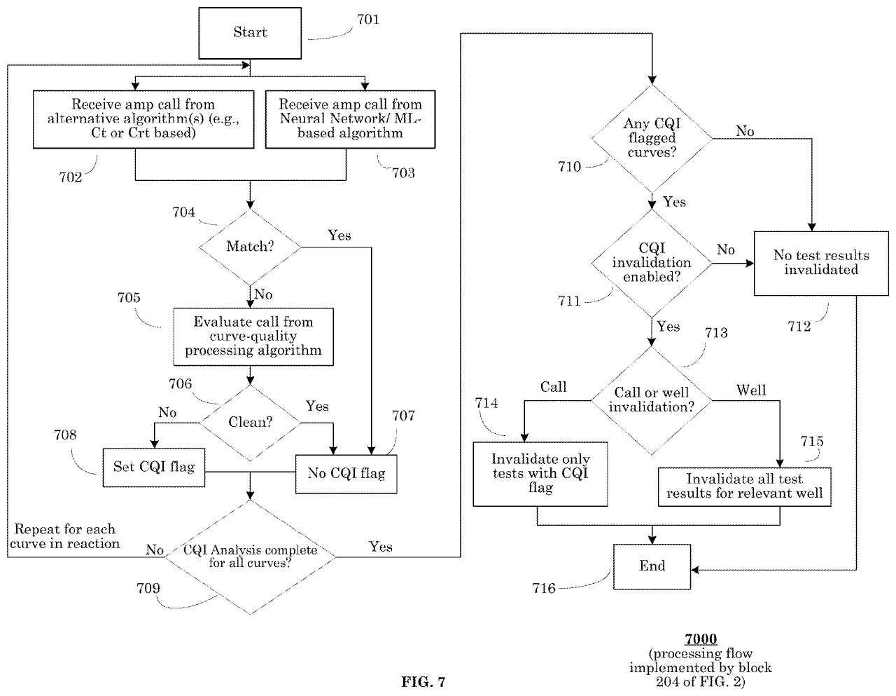

As will be further described below in the context of FIG. 7, evaluation block 205 receives amplification call and curve-quality call information and uses it to evaluate which neural network-based amp calls, if any, should be invalidated. In some embodiments, indications that a neural network (or other ML-based) amp call should be invalidated are based on user-selected settings in an electronic graphical user interface (GUI). In some embodiments, the GUI allows users to selectively invalidate calls based on visual inspection of a corresponding amplification curve flagged for review by evaluation block 205. In some embodiments, the GUI allows users to set parameters (such as confidence requirements, curve-quality probability requirements, and/or other requirements) that evaluation block 205 uses to determine which neural network-based amp calls should be invalidated and/or flagged for further review by a user.

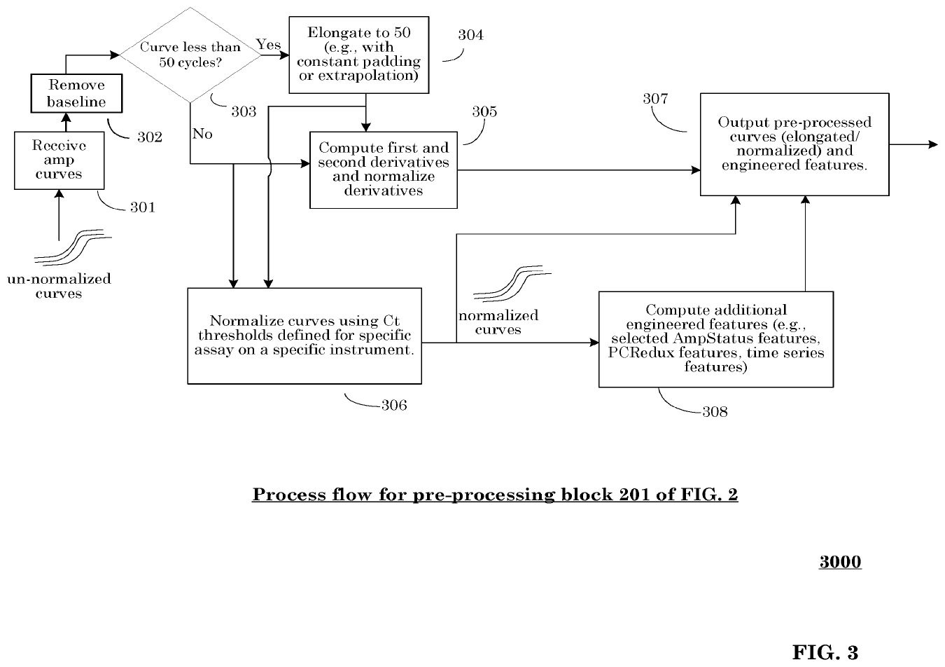

FIG. 3 illustrates pre-processing 3000 implemented by pre-processing block 201 of FIG. 2 in one embodiment of the present disclosure. Step 301 receives amplification curves obtained from, for example, qPCR assays run on a qPCR instrument. Step 302 removes a baseline from the curves that, in this example, corresponds to a baseline signal generated by the instrument in the absence of a template. The baseline contains noise before a sufficient signal to noise ratio is reached. One skilled in the art will appreciate that there are various ways baseline estimation can be carried out. In one embodiment, the baseline is estimated by linear least squares through the points of the curve that is chosen as the baseline region. In one embodiment, the baseline is estimated by the average of the points of the curve that is chosen as the baseline region. Step 303 determines if a received amp curve is less than 50 cycles. If yes, the processing proceeds with step 304 elongating the data to 50-cycle data. One skilled in the art will appreciate various ways elongation can be carried out. In one embodiment, constant padding is used, which simply involves repeating the last cycle value (e.g., a value corresponding to a 40th cycle) until the data is 50 cycles long. The elongated 50-cycle curve resulting from step 304 is provided to derivative-computing step 305 and to normalizing step 306. If the result of step 303 is no, then the 50-cycle curve is provided without elongation directly to step 305 and step 306. In alternative embodiments, the data can be standardized to 40-cycle data and 50-cycle or other larger-than-40-cycle curves can be truncated or interpolated to obtain 40-cycle data as described, for example, in U.S. Application Ser. No. 17,346,147, filed Jun. 11, 2021, publication number US2021/0391033, the entire of contents of which is hereby incorporated by reference.

In the illustrated embodiment, derivative-computing step 305 computes derivatives from an unnormalized amplification curve of 50 qPCR cycles. It computes both a 1^{st} order derivative and a 2^{nd} order derivative. In one example, the derivative computations are performed using a Savitzky-Golay filter and the following Savitzky-Golay parameters: Polynomial Order=3; and Window Length=9. The 1st and 2nd order derivatives may be normalized using max-normalization. The resulting normalized derivative values represent a portion of the engineered features output by pre-processing 3000.

Step 306 normalizes the amp curves using cycle threshold (“Ct”) values for a specific assay on a specific instrument. The normalization may be performed by dividing the each value of the unnormalized curve with the Ct value corresponding to the assay from which the curve was obtained. In alternative embodiments, different values can be used for normalizing.

Normalized curves are used by step 308 to compute additional engineered features. In one embodiment, additional engineered features include, for example, one or more of the following: Features related to the Cycle Relative Threshold (“Crt”) algorithm, a version of which is described in patent publication US 2016/0110495 A1 of U.S. patent application Ser. No. 14/921,948 (“Crt patent publication”); features from PCRedux (see PCRedux: A Data Mining and Machine Learning Toolkit for qPCR Experiments at biorxiv.org/content/10.1101/2021.03.31.437921v1); time series features (e.g., shapelets, wavelets); and features associated with various anomalies (e.g., waterfall, bubble, wavy baseline, and creep). Both of the publications referenced above are hereby incorporated by reference in their entireties.

In one embodiment, one or more of the features from the Crt algorithm listed and described in Table 1 below are computed and used. Cross references to the above-referenced Crt patent publication, where applicable, are also provided below:

TABLE 1

| Crt Features | Description/Formula | Crt Pat Pub. Par. |
|---|---|---|
| AmpStatus | AmpStatus Predictions: {1: AMP, 0: UNK, −1: NON} by Crt | |
| | algorithm. See below this table for further explanation. | |
| effRanged | Actual range of the baselined amplification curve multiplied | |
| | by the Crt Amp Factor (see “alphaAmp”, last row below) | |
| ampAdjusted | The Crt Amp Factor divided by RMS (root mean square) noise. | Referred to as noise- |
| InverseRMSNoise | | normalized |
| | | amplification curve |
| | | amplitudes. E.g. |
| | | [0014] |
| adjustedXShift | adjustedXShift = XShift + XAdjust. | Called the Half- |
| | XShift: Refers to a (proxy) horizontal shift in the | Efficiency Cycle. E.g. |
| | maxima of the second derivative of the amplification | [0052] |
| | curve. | |
| | XAdjust: Adjustment in efficiency curve to accounts for | |
| | whether the efficiency curves start at a value of 1. | |
| equivDilutionSeries | Efficiency at the maxima of the second derivative of the | |
| Effics | amplification curve. (Note: The modeled efficiency at the | |
| | calculated Crt cycle) | |
| efficExponent | Decides the slope of the Crt modeled efficiency curve and | Referred to as |
| | correspondingly the Crt modeled amplification curve | alphaBend. E.g. |
| | | [0049] |
| CACs | Cycle Absolute Copy - determines the absolute genetic | See par. [0078] |
| | quantification or copy number based on single amplification | |
| | curve. CAC is a quantification metric that is based on | |
| | equivalent Cq cycle for fixed number of copies to be reached if | |
| | the qPCR efficiency could have been kept constant at 1. It has | |
| | the property that a CAC increase of 1 represents a precise | |
| | halving of the initial copy number. | |
| normalizedWeighted | A weighted RMS (root mean square) error calculation | Model Curve Fit. E.g. |
| RMSPredictionError | determined by Crt algorithm. | [0083] |
| alphaAmp | A Crt parameter that represents the modeled amplitude | Normalized modeled |
| | fraction for each (0-1) normalized amp curve. This is also | curve amplitude. E.g. |
| | called the Crt Amp Factor. | [0055] |

“AmpStatus” is a prediction result of the Crt algorithm. It can be obtained using software in commercially available products from Thermo Fisher Scientific including, among others, one or more of the following products: QuantStudio™ 12k Flex v1.5; QuantStudio™ 6 and 7 Flex v1.7.2; Relative Quantification v4.3 (Cloud App); Standard Curve v4.0 (Cloud App); and Presence Absence Analysis v1.9 (Cloud App).

In one embodiment, one or more of the PCRedux features listed in Table 2 are determined and used:

TABLE 2

| PCRedux Feature | Description/Formula |
|---|---|
| Cor | value of the correlation coefficient from a Pearson linear correlation analysis between |
| | all PCR cycles and the fluorescence signals. |
| window_Win_1 to | the complete curve trajectory is segmented into 10 equidistant windows by fitting an |
| window_Win_10 | interpolating smoothing spline with smoothing factor 0.5 to the curve, interpolating |
| | exactly 50 curve points, and then cutting these into 10 windows of five values each, |
| | with a subsequent calculation of the MAD/Median ratio for each of these windows. |
| mindful | minimum of fluorescence amplitude |
| hooreg_hook_slope | estimate of slope of the Hook effect like curvature |
| fitAIC | value of the Akaike's second-order corrects Information Criterion, which was |
| | determined on a non-linear (sigmoid) four-parameter model |
| head2tail_ratio | ratio between the signal of the background and tail regions |
| mblrr_slope_pt | the value of the slope in the estimated plateau phase of the amplification curve |
| mblrr_cor_pt | the value of the linear correlation coefficient in the estimated plateau phase of the |
| | amplification curve |
| mblrr_slope_bg | the value of the slope in the estimated background region of the amplification curve |
| mblrr_cor_bg | the value of the intercept in the estimated background region of the amplification |
| | curve |
| cpD1 | maximum of the first derivative curve |
| cpD2 | maximum of the second derivative curve |
| cpD2_range | cycle difference between the maximum and the minimum of the second derivative |
| | curve |
| loglin_slope | slope determined by a linear model of the data points from the minimum and |
| | maximum of the second derivative |
| polyarea | area of a polygon given by the vertices in the vectors cycles and fluorescence |
| sumdiff | describes proportion of cycles x in which the fluorescence signal of x is |
| | smaller than in x + 1 |
| direction | test if the maximum of the first derivative is positive or negative |
| peaks_ratio | uses the estimate of approximate local minimums and maximums |
| cp_e.agglo | agglomerative hierarchical estimate for multiple change points |
| cp_bcp | change point by Bayesian analysis methods |
| sd_bg | shows the standard deviation of the fluorescence in the ground phase |

In one embodiment, step 308 normalizes the additional engineered features after computing them. In one embodiment, a quantile normalizer is used. A quantile normalizer transforms the features to follow a normal distribution. The number of quantiles that are computed is equal to 1000. A scaler to be used for normalizing is obtained from a chosen representative training set for a given model.

Step 307 outputs the normalized derivatives generated by step 305, the normalized curves generated by step 306, and the additional normalized engineered features generated by step 308. These values are output by, for example, pre-processing block 201 shown in FIG. 2, and are used by various parts of the amplification analysis system as further described below.

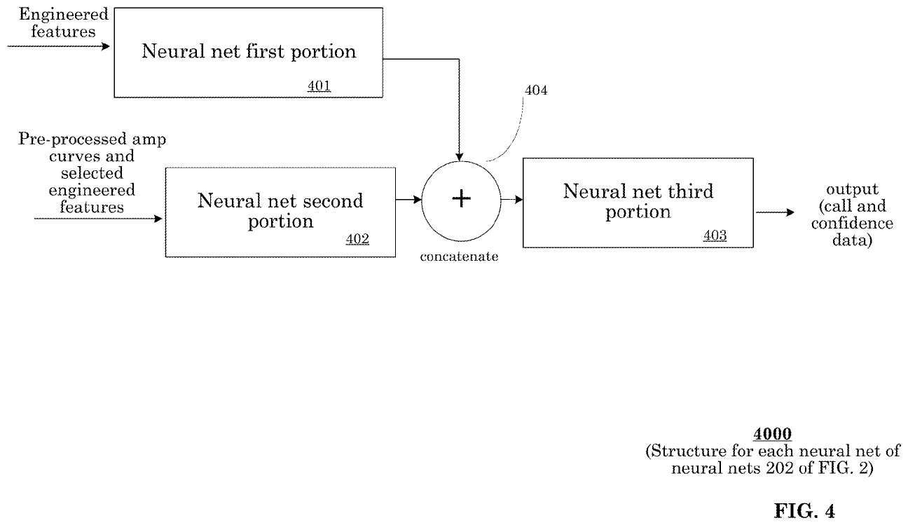

FIG. 4 shows a high-level block diagram of an architecture 4000 of one of amp-calling neural network(s) 202 of FIG. 2 according to one embodiment of the disclosure. Note that, in one embodiment, amp-calling network(s) 202 use an ensemble approach including a plurality (e.g., 4, 5, or some other number) of similarly structured but differently trained neural networks. Various techniques can be used for combining results of individual networks in an ensemble (e.g., averaging, majority-voting, etc.) to return class probabilities for a given curve generated from a particular assay on a sample. Also, various techniques can be used to differently train the networks of an ensemble (e.g., using different training data, ordering the training data differently, initializing weights/filters to different values, etc.). FIG. 4 shows the structure used for each neural network of an ensemble of neural networks 202, according to one embodiment of the disclosure.

Structure 4000 comprises neural net (“artificial,” i.e., computerized, neural network implied throughout when referencing a “neural net” or “neural network” herein; other deep learning networks might be used in alternative embodiments, and “deep learning network” as used herein will also be assumed to imply “computerized deep learning network”) first portion 401, neural net second portion 402, and neural net third portion 403. In the illustrated embodiment, neural net second portion 402 receives (from pre-processing block 201 of FIG. 2) pre-processed amp curves along with selected engineered features. In this example, the selected engineered features received by neural net second portion 402 include the normalized first and second order derivatives computed at step 305, as previously shown and described in the context of FIG. 3. However, in alternative embodiments, other engineered features might be received by second portion 402 in addition to or instead of derivative values, or, in some embodiments, the pre-processed amp curves might be received and used by second portion 402 without any engineered features.

Additional engineered features are received by neural net first portion 401. In the illustrated embodiment, the additional engineered features include one or more of the features previously described in the context of step 308 of FIG. 3 (e.g., Crt algorithm features, PCRedux features, time series features, known anomaly features, etc.). However, in alternative embodiments, different engineered features can be received by neural net first portion 401.

Neural net first portion 401 and neural net second portion 402 process their respective inputs. Their respective outputs are joined by concatenation function 404, which provides the concatenated output as the input to neural net third portion 403. Neural net third portion 403 processes the concatenated output of first portion 401 and second portion 402 and generates, as output, call and confidence data. In an alternative embodiment, only amplification call data is determined and output by neural net third portion 403.

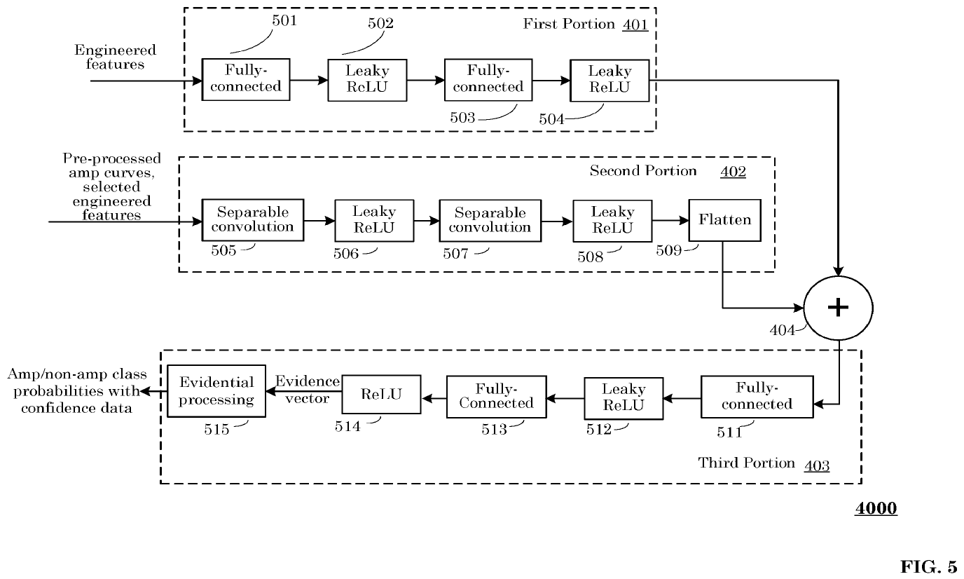

FIG. 5 is a block diagram illustrating the neural network structure 4000 of FIG. 4 in further detail in accordance with one embodiment of the present disclosure. As shown, neural net first portion 401 comprises a fully-connected layer 501, a Leaky ReLU layer (activation function) 502, fully-connected layer 503 and a Leaky ReLU layer (activation function) 504. Neural net second portion 402 comprises separable convolution layer 505, Leaky ReLU layer (activation function) 506, separable convolution layer 507, Leaky ReLU layer (activation function) 508, and flattening layer 509. As described above in the context of FIG. 4, concatenation operation 404 concatenates the output of first portion 401 and second portion 402 and provides the concatenated output to the input of third portion 403. Third portion 403 comprises fully connected layer 511, Leaky ReLU layer (activation function) 512, fully connected layer 513, ReLU layer (activation function) 514, and evidential processing module 515.

The above structures will now be describe with further details regarding layer input/output sizes and other characteristics of a particular embodiment. The disclosed details have been found to work well for the purpose of the amplification curve analysis application described herein. But one skilled in the art will understand that many of these details can be varied from those referenced below without necessarily departing from the spirit and scope of the present disclosure.

In one embodiment, fully-connected layer 501 has 9-unit (1×9) input data and 16-unit (1×16) output data. Leaky ReLU layer 502 is an activation function operating on fully-connected layer 501's output and it provides 1×16 output to fully connected layer 503 that, in turn, provides 1×16 output that is processed by Leaky ReLU layer 504 to provide 1×16 output to concatenation function 404.

Turning to further details of neural net second portion 402, separable convolution layer 505, in this embodiment, receives 3-channel data, each channel including 50 units, i.e., 3×50 data. Specifically, for a given cycle of the 50-cycle amp curve data, three values are provided as input to convolution layer 505: the pre-processed amp curve's normalized fluorescence value at that cycle, the first order derivative, and the second order derivative.

In the illustrated embodiment, separable convolution layer 505 uses 16 filters, each 3×3 in size. A stride of 2 is used and sufficient padding is applied to the input array to obtain the desired output feature map dimensions which, for separable convolution layer 505, are 16×26. In this embodiment, that means that the 3×50 input array is separated into three “sub” arrays, each being 1×50 in size. Similarly, each 3×3 filter is separated into three “sub” filters, each being 1×3 in size. Each respective sub-array is convolved with a respective sub-filter to create a respective 1D feature map. The resulting feature maps are “stacked” to provide a 2D feature map. A pointwise convolution is performed on the 2D feature map in which it is convolved with a 1×3 filter. This pointwise step outputs a 1×26 array, which is the same size feature map as would have resulted from a normal convolution of 3×3 filter with a 50×3 input array, including necessary padding. With 16 filters used in the illustrated embodiment, the resulting output from separable convolutional layer 505 is a 16×26 feature map.

In alternative embodiments, more or fewer filters can be used in each of the separable convolutional layers described herein. The number of filters shown in the presently illustrated embodiment is preferred, but can be different in alternative embodiments without necessarily departing from the spirit and scope of the present disclosure. In the illustrated embodiment, separable convolution layer 505 uses a stride of 2 and uses replication padding. Replication padding makes a copy of a sequence in the input array to be padded, reverses it, and then uses the reversed sequence to pad on either end of the sequence. Padding allows the convolution process to create feature maps that have the desired length dimension. Alternative embodiments use different types of padding, e.g. “same” padding, without necessarily departing from the spirit and scope of the disclosure.

Other separable convolutional layers in the illustrated embodiment use the same filter size, stride, padding type, and depth-wise followed by point-wise convolution; those characteristics will be assumed and not repeated further below, though the characteristics can be varied in alternative embodiments.

The resulting 16×26 output from separable convolutional layer 505 is processed by a Leaky ReLU activation function, represented here by Leaky ReLU layer 506, and the results are provided as 16×26 data to separable convolution layer 507. Separable convolution layer 507 uses 8 filters. The resulting 8×13 output data is processed by Leaky ReLU layer 508, and the results are provided as 8×13 data to flattening layer 509. Flattening layer 509 converts the 8×13 data to single-column (i.e., one dimensional) array of length 104, and provides the resulting 1×104 output to concatenation operation 404.

The 1×16 output of Leaky ReLU 504 of the first portion 401 and the 1×104 output of the flattening layer 509 of the second portion 402 are concatenated at block 404 and the concatenated resulting 1×120 data is provided to fully connected layer 511 of the third portion 403.

Fully-connected layer 511 receives the 1×120 concatenated data and provides 1×16 output data, which is processed by Leaky ReLU layer 512 which in turn provides 1×16 output to fully connected layer 513, which in turn provides output from a 2-node final layer that provides 2-unit output (the output dimension corresponding to the number of classifications for the illustrated embodiment, i.e., two classifications: amplified and non-amplified).

In the illustrated embodiment, the output of fully-connected layer 513 is processed by ReLU layer 514. The illustrated embodiment implements evidential learning. The output of ReLU layer 514 is used as an evidence vector by evidential processing block 515. Evidential processing block 515 uses evidential learning techniques to process the received evidence vector and obtain class probability determinations corresponding to amp and non-amp classifications for each amplification curve along with confidence measurements for the corresponding classification data. An example of evidential learning techniques used by block 515 in the illustrated embodiment are described in, Sensoy et al., “Evidential Deep Learning to Quantify Classification Uncertainty,” arXiv:1806.01768v3 [cs.LG] 31 Oct. 2018, https://arxiv.org/pdf/1806.01768.pdf, incorporated herein by reference in its entirety.

In an alternative embodiment, ReLU layer 514 and evidential processing block 515 are replaced by a softmax layer that simply converts the output of fully connected layer 513 to class probabilities and does not generate confidence data.

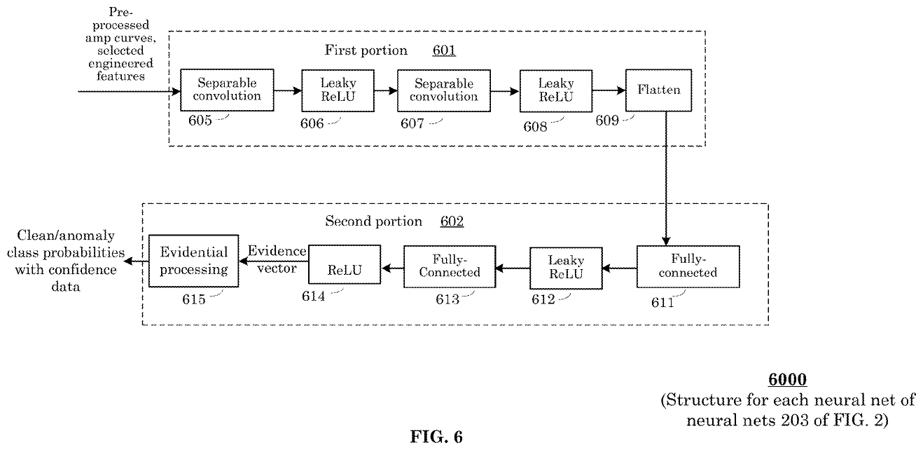

FIG. 6 is a block diagram of an architecture 6000 of one of curve-quality calling neural network(s) 203 of FIG. 2 according to one embodiment of the disclosure. Note that, in one embodiment, curve-quality calling network(s) 203 uses an ensemble approach including a plurality (e.g., 4, 5, or some other number) of similarly structured but differently trained neural networks (as previously described in the context of amp-calling neural network(s) 202). FIG. 6 shows the structure used for each neural network of an ensemble of neural networks 203, according to one embodiment of the disclosure.

In the illustrated embodiment, the same pre-processed amp curves and selected engineered features (first and second order derivatives) submitted to neural net second portion 402 (see FIGS. 4 and 5 and accompanying text) are also submitted to first portion 601 of the curve-quality calling neural net illustrated in FIG. 6. In alternative embodiments, only the pre-process amp curves are submitted. In other alternative embodiments, one or more additional engineered features are also submitted along with the pre-processed amp curves.

Neural net first portion 601 can be understood as operating to extract learned features from the pre-processed amp curves (along with selected engineered features of those curves) that it receives. The optimized parameter values (e.g., convolutional filter values) used to extract those features from each curve are learned during training of the neural network. Second portion 602 can be understood as a classification network used to classify curves as “clean” or “problem.”

As shown, neural net first portion 601 comprises separable convolution layer 605, Leaky ReLU layer 606, separable convolution layer 607, Leaky ReLU layer 608, and flattening layer 609. In the illustrated embodiment, the more detailed characteristics (e.g., input/output data dimensions, number of filters, filter size, stride, padding type) of separable convolution layer 605, Leaky ReLU layer 606, separable convolution layer 607, Leaky ReLU layer 608, and flattening layer 609 are the same as previously described for, respectively, separable convolution layer 505, Leaky ReLU layer 506, separable convolution layer 507, Leaky ReLU layer 508, and flattening layer 509, and those description are therefore not repeated here.

Thus, the input provided from flattening layer 609 to fully-connected layer 611 is 1×104 in size. Fully connected layer 611 outputs 1×16 data for processing by Leaky ReLU layer 612. The resulting 1×16 data is input to fully connected layer 613, which outputs 1×2 data to ReLU layer 614. The resulting evidence vector is processed by evidential processing block 615 in a manner similar to that described above in the context of evidential processing block 515. Evidential processing block 615 outputs class probabilities and corresponding confidence measurements regarding the following curve-quality classifications: “clean” or “problem.”

In an alternative embodiment, ReLU layer 614 and evidential processing block 615 are replaced by a softmax layer that simply converts the output of fully connected layer 613 to class probabilities and does not generate confidence data.

FIG. 7 illustrates processing flow 7000 implemented by amp and curve-quality call evaluation block 205. Processing flow 7000 receives amp call data and curve-quality call data generated by curve analysis algorithms such as those previously described herein. Processing flow 7000 flow provides one example of a processing flow for curve-analysis results processing that can be used to support presentation of results to a user interacting with a graphical user interface (GUI) on a user device implementing, or that is coupled to other computers implementing, embodiments of the curve-analysis systems consistent with the present disclosure.

Processing 7000 being at step 701. Step 702 receives amp calls from alternative amp calling algorithms such as one or more of those previously described (or from other alternative amp-calling algorithms). Step 703 receives amp calls from a neural network/machine-learning based algorithm such as, for example, neural network(s) 202 of FIG. 2. Step 704 determines whether the results received at steps 702 and 703 match. If yes, then processing proceeds to step 707 and no curve quality indicator (CQI) flag is set. If the result of step 704 is no, then step 705 evaluates call received from a curve-quality processing algorithm such as, for example, neural networks 203 of FIG. 2. Step 706 determines if the curve-quality call was “clean” (i.e., not “problem”). If no, then processing proceeds to step 708 and a CQI flag is set. If yes, then processing proceeds to step 707 and no CQI flag is set for that curve. In either case, processing proceeds to step 709 which determines whether CQI analysis is complete for all curves generated from a particular sample well whose curve(s) that are generated for one or more tests are currently being analyzed. If the result of step 709 is no, then processing returns steps 702 and 703 for a next curve. If the result of step 709 is yes, then processing proceeds to step 710.

Step 710 determines if any curves corresponding to the currently analyzed well have CQI flags. If no, then processing proceeds to step 712 and 716 directly and none of the tests associated with the current well being analyzed are invalidated. If yes, then processing proceeds to step 711 to determine if CQI invalidation is currently enabled. In some embodiments, this is a user-selected setting. In alternative embodiments, whether CQI invalidation is enabled can be automatically determined based on various user-selected or pre-determined factors and/or can be enabled by default. If CQI invalidation is not enabled, then the result of step 711 is no and processing proceeds to step 712 and 716 and none of the tests associated with the current well being analyzed are invalidated. If CQI invalidation is enabled, then the result of step 711 is yes and processing proceeds to step 713 to determine whether calls only from individual tests associated with CQI flags in a given well should be invalidated or whether all calls for tests in the well should be invalidated. If the result of step 713 is “call,” then only test results associated with CQI flags are invalidated. If the result of step 713 is “well,” then all results for that well are invalidated, whether or not the individual test is associated with a CQI-flagged amplification curve.

Those skilled in the art will appreciate that, in various embodiments, different factors can be used to determine whether to invalidate all tests in a well or only ones associated with calls for CQI-flagged curves. In some embodiments, this determination can be made from available data based on user-set criteria. For example, a user might set criteria based on the number or percentage of CQI-flagged calls in a given well to determine whether or not to invalidate all calls in the well. Once the user-based criteria is set, then the result can be implemented automatically based on the call and CQI flag data. If confidence measurements are available, then a user could, in some embodiments, set criteria based on confidence scores meeting or not meeting certain thresholds at the level of individual calls and/or across all calls in the well. Conditional criteria based on various combinations of call and curve quality data could also be implemented. Various alternatives consistent with the present disclosure will be apparent to one skilled in the art.

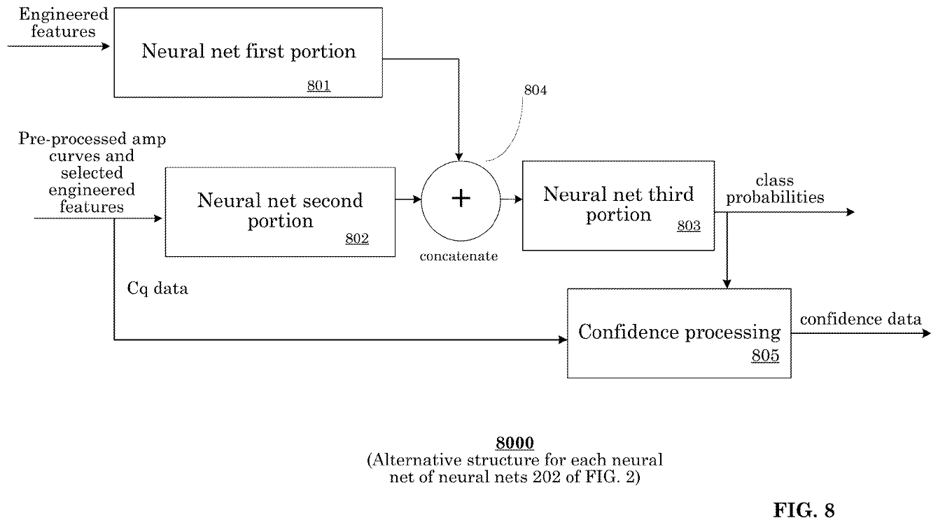

FIG. 8 illustrates a high-level block diagram of an alternative architecture 8000 of one of amp-calling neural network(s) 202 of FIG. 2 according to one embodiment of the disclosure. The illustrated structure 8000 is an alternative to the structure 4000 illustrated in FIG. 4.

As previously described in the context of FIG. 4, in one embodiment, amp-calling network(s) 202 use an ensemble approach including a plurality (e.g., 4, 5, or some other number) of similarly structured but differently trained neural networks. Various techniques can be used for combining results of individual networks in an ensemble (e.g., averaging, majority-voting, etc.) to return class probabilities for a given curve generated from a particular assay on a sample. Also, various techniques can be used to differently train the networks of an ensemble (e.g., using different training data, ordering the training data differently, initializing weights/filters to different values, etc.). FIG. 8 shows the structure used for each neural network of an ensemble of neural networks 202, according to one alternative embodiment of the disclosure.

Structure 8000 comprises neural net first portion 801, neural net second portion 802, and neural net third portion 803. In the illustrated embodiment, neural net second portion 802 receives (from pre-processing block 201 of FIG. 2) pre-processed amp curves along with selected engineered features. In this example, the selected engineered features received by neural net second portion 802 include the normalized first and second order derivatives computed at step 305, as previously shown and described in the context of FIG. 3. However, in alternative embodiments, other engineered features might be received by neural net second portion 802 in addition to or instead of derivative values, or, in some embodiments, the pre-processed amp curves might be received and used by neural net second portion 802 without any engineered features.

Additional engineered features are received by neural net first portion 801. In the illustrated embodiment, the additional engineered features include one or more of the features previously described in the context of step 308 of FIG. 3 (e.g., Crt algorithm features, PCRedux features, time series features, known anomaly features, etc.). However, in alternative embodiments, different engineered features can be received by neural net first portion 801.

Neural net first portion 801 and neural net second portion 802 process their respective inputs. Their respective outputs are joined by concatenation function 804, which provides the concatenated output as the input to neural net third portion 803. Neural net third portion 803 processes the concatenated output of neural net first portion 801 and neural net second portion 802 and generates, as output, call amplification call data. In one example, such call data comprises class probabilities (sometime referred to herein as prediction scores) for one or more classifications such as amplified and/or not amplified.

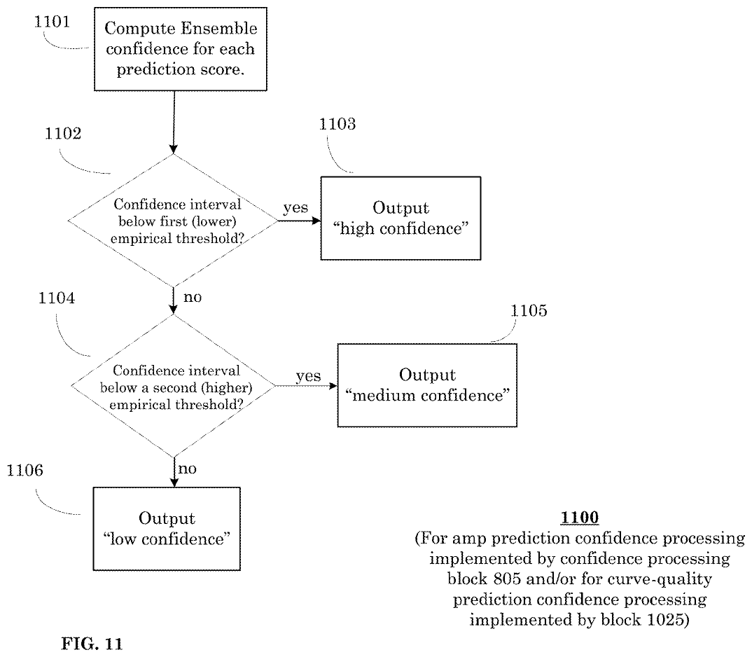

As further shown in FIG. 8, the class probabilities output by neural net third portion 803 are also provided to confidence processing block 805. Confidence processing block 805 also receives quantification cycle (Cq) data (sometimes referred to as cycle threshold, or Ct data) including a Cq value for each amplification curve for which amp class probabilities are generated. Confidence processing block 805 generates confidence data. One embodiment for implementing the processing of confidence processing block 805 is further described below in the context of FIG. 11.

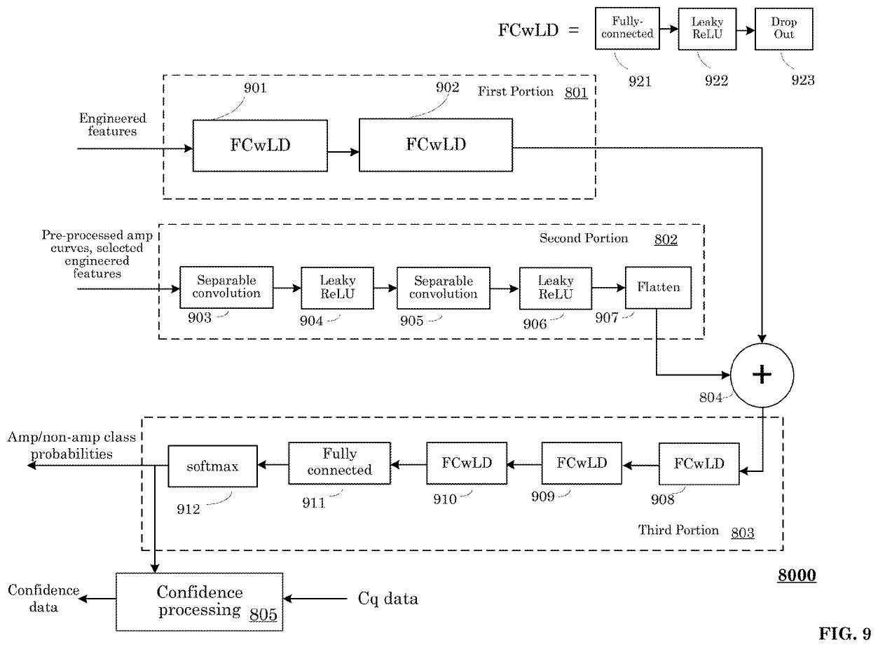

FIG. 9 is a block diagram illustrating the neural network structure 8000 of FIG. 8 in further detail in accordance with one embodiment of the present disclosure. As shown, neural net first portion 801 comprises FCwLD processing blocks 901 and 902. Each FCwLD processing block comprises a fully connected layer 921 followed by a Leaky ReLU layer (activation function) 922, and a dropout layer 923. Neural net second portion 802 comprises separable convolution layer 903, Leaky ReLU layer (activation function) 904, separable convolution layer 905, Leaky ReLU layer (activation function) 906, and flattening layer 907. As described above in the context of FIG. 8, concatenation operation 804 concatenates the output of neural net first portion 801 and neural net second portion 802 and provides the concatenated output to the input of neural net third portion 803. Neural net third portion 803 comprises FCwLD blocks 908, 909, 910 followed by fully connected layer 911 and softmax layer 912. Like FCwLD blocks 901 and 902, FCwLD blocks 908, 909, 910 each comprise fully a connected layer 921 followed by a Leaky ReLU layer 922, and a dropout layer 923.

In the illustrated embodiment, each dropout layer 923 in an FCwLD block uses a dropout rate of 0.5. However, other rates can be used without necessarily departing from the spirit and scope of the present disclosure. In the illustrated embodiment, separable convolution layers 903 and 905 operate in a manner similar to that described for separable convolution layers 505 and 507 in FIG. 5.

In the illustrated embodiment, softmax layer 912 outputs class probabilities (prediction scores) for use by a computer user interface and for use by confidence processing block 805.

In an alternative embodiment, softmax layer 912 and confidence processing block 805 are replaced by a Leaky ReLU layer and evidential learning block as previously described that generate amplification call data and confidence data.

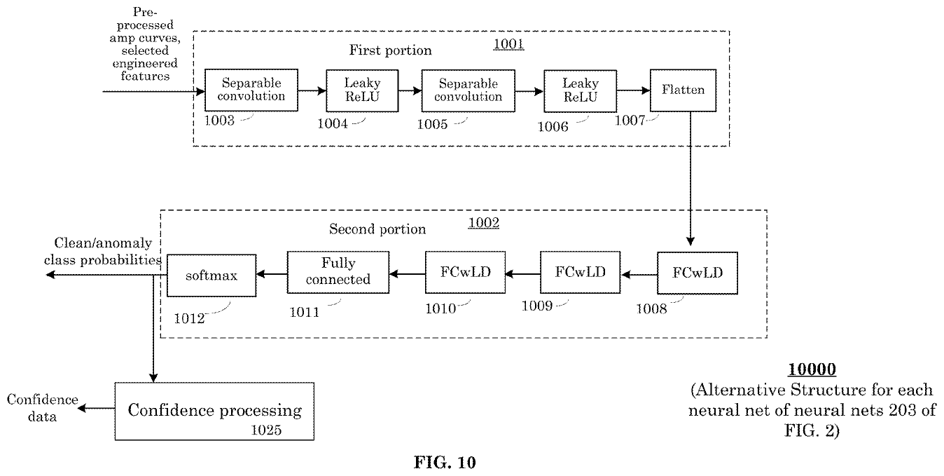

FIG. 10 illustrates a high-level block diagram of an alternative architecture 10000 of one of curve-quality calling neural network(s) 203 of FIG. 2 according to one embodiment of the disclosure.

As shown, neural net first portion 1001 comprises separable convolution layer 1003, Leaky ReLU layer (activation function) 1004, separable convolution layer 1005, Leaky ReLU layer (activation function) 1006, and flattening layer 1007. Neural net second portion 1002 comprises FCwLD blocks 1008, 1009, and 1010 followed by fully connected layer 1011 and softmax layer 1012. Like FCwLD block 901 and 902, FCwLD blocks 1008, 1009, and 1010 each comprise a fully connected layer 921 followed by a Leaky ReLU layer 922, and a dropout layer 923.

In the illustrated embodiment, each dropout layer 923 in an FCwLD block uses a dropout rate of 0.5. However, other rates can be used without necessarily departing from the spirit and scope of the present disclosure. In the illustrated embodiment, separable convolution layers 1003 and 1005 operate in a manner similar to that described for separable convolution layers 605 and 607 in FIG. 6.

In the illustrated embodiment, softmax layer 1012 outputs class probabilities (prediction scores) for use by a computer user interface and for use by confidence processing block 1025.

In an alternative embodiment, softmax layer 1012 and confidence processing block 10255 are replaced by a Leaky ReLU layer and an evidential learning block as previously described that generate curve-quality call data and confidence data.

FIG. 11 is a flow diagram illustrating processing 1100 carried out by amp prediction confidence processing block 805 shown in FIGS. 8 and 9 in accordance with an embodiment of the present disclosure. Similar processing steps can be used by curve-quality prediction confidence processing block 1025 shown in FIG. 10. Processing 1100 will first be described in the context of amp-prediction confidence processing carried out by block 805.

Processing 1100 begins at step 1101, which computes ensemble confidence for each prediction score. As previously explained, some embodiments of the disclosure use an ensemble of deep learning networks that are similarly structured but are differently trained. In relevant embodiments using processing 1100, each deep learning amp-calling network 202 in an ensemble of such networks generates a prediction score for a particular amp curve. As one skilled in the art will appreciate, various statistical techniques can be used to generate a confidence metric using set of data points drawn from an underlying population.

In some embodiments, the 95% confidence interval is computed for an ensemble prediction score using all the individual prediction scores generated by the amp-calling networks 202 in the ensemble and assuming a normal distribution. Then, the difference between the upper and lower bounds of that confidence interval is used as a confidence metric and empirical thresholds are used to assign and output an intuitive confidence level to the prediction score result (e.g., “low,” “medium,” or “high” confidence). For example, a confidence interval for an ensemble's prediction scores might have a lower bound (LB) of 0.75 and an upper bound (UB) of 0.85. The resulting confidence metric (UB-LB) would be 0.10. Using, for example, a threshold of 0.15 such that anything below 0.15 is “high” confidence, the process would assign a high confidence level to the corresponding amp prediction score of the ensemble as a whole (the ensemble's overall prediction score might, for example, be taken as the average prediction score of the all the scores generated for the given curve by the amp-calling networks of the ensemble).

Step 1102 determines whether the computed confidence metric is below a first, lower threshold. If the result of step 1102 is yes, then processing proceeds to step 1103 which outputs a “high” confidence level. If the result of step 1102 is no, then processing proceeds to step 1104 which determines whether the confidence metric (e.g., UB-LB of the confidence interval) is below a next, higher threshold. If the result of step 1104 is yes, then processing proceeds to step 1105 which outputs a “medium” confidence level. If the result of step 1104 is no, then processing proceeds to step 1106 which outputs a “low” confidence level.

In one embodiment, the following thresholds are used: If the UB-LB of the 95% confidence interval is less than 0.25, then confidence is determined to be “high.” If UB-LB of the 95% confidence interval is equal to or greater than 0.25, but less than 0.6, then the confidence level is determined to be “medium.” If UB-LB of the 95% confidence interval is 0.6 or greater, then the confidence level is determined to be “low.” However, one skilled in the art will appreciate that appropriate thresholds for confidence determinations will be dependent on the data sets and context. And different thresholds might be used depending on the statistics of the underlying data set.

Also, the thresholds used might change based on what Cq value is associated with the amplification curve being processed for prediction. For example, over some Cq ranges, a tighter range of prediction scores might be expected. As one example, if the Cq value corresponds to a cycle in the range of 12-20, then the amplified class probability would be expected to be consistently high (near 1) and a tighter confidence interval range (lower threshold for UB-LB) might be required (e.g., 0.2 or 0.15 instead of 0.25) in order to assign a “high” confidence value to the class probability (prediction score). Therefore, in some embodiments, a first (lower) threshold is dependent on Cq value. For example, a first threshold might be used at step 1103 that is lower than 0.25 (e.g., 0.15, 0.2) when the corresponding amplification curve has a relatively low Cq value (e.g., 12-20) and 0.25 or some other value higher than 0.2 might be used for curves with higher Cq values. In some embodiments, a second (higher) threshold is also dependent on Cq values.

In some embodiments, processing 1100 (or similar processing) is used by block 1025 of FIG. 10 to assign confidence values to predictions scores for curve-quality predictions. For curve-quality predictions, the appropriate thresholds for assigning low, medium, or high confidence will also be dependent on particular data sets and context. But the processing might be similar as is described above in the context of amp-prediction confidence processing. For example, in some-embodiments using processing 1100 for curve-quality prediction confidence processing, the 95% confidence interval is computed for an ensemble prediction score using all the individual prediction scores generated by the curve-quality calling networks 203 in the ensemble and assuming a normal distribution. Then, the difference between the upper and lower bounds of that confidence interval is used as a confidence metric and empirical thresholds are used to assign an intuitive confidence level to the prediction score result (e.g., “low,” “medium,” or “high” confidence). In some embodiments, 0.25 is used as a first (lower) threshold and if the ensemble confidence metric is below 0.25, a “high” confidence is assigned. If the ensemble confidence metric is equal to or greater than 0.25 but below 0.6, then a “medium” confidence is assigned. If the ensemble confidence metric is not below 0.6, then a “low” confidence is assigned.

Although Cq values might be less relevant to assessing confidence of curve-quality predictions than they are to assessing confidence of amplification predictions, in some embodiments, other context-dependent factors might be used to modify thresholds for curve-quality confidence processing. As noted above, one skilled in the art will appreciate that appropriate thresholds for assigning confidence levels can be dependent on the context of a particular data set. The above thresholds are set forth merely by way of example.

As one skilled in the art will appreciate, in alternative embodiments, the confidence metric itself and/or other underlying statistics regarding the relevant dataset can be output through a computer user interface without necessarily assigning intuitive confidence values such as “low,” “medium,” or “high.” A sophisticated user might prefer to assess confidence based on the underlying confidence interval metrics rather than having the assessment generated automatically.

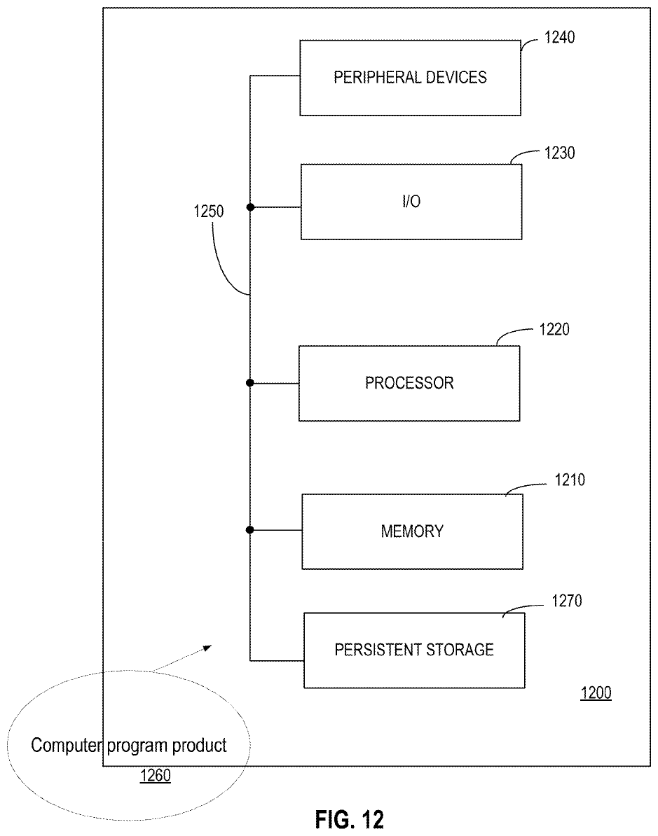

FIG. 12 illustrates an exemplary computer system configurable by a computer program product to carry out embodiments of the present invention.

In the example, computer system 1200 may provide one or more of the components of an automated qPCR curve analysis system configured to implement one or more logic modules and artificial neural networks and associated components for a computer-implemented qPCR automated analysis system and associated interactive graphical user interface. Computer system 1200 executes instruction code contained in a computer program product 1260. Computer program product 1260 comprises executable code in an electronically readable medium that may instruct one or more computers such as computer system 1200 to perform processing that accomplishes the exemplary method steps performed by the embodiments referenced herein. The electronically readable medium may be any non-transitory medium that stores information electronically and may be accessed locally or remotely, for example, via a network connection. In alternative embodiments, the medium may be transitory. The medium may include a plurality of geographically dispersed media, each configured to store different parts of the executable code at different locations or at different times. The executable instruction code in an electronically readable medium directs the illustrated computer system 1200 to carry out various exemplary tasks described herein. The executable code for directing the carrying out of tasks described herein would be typically realized in software. However, it will be appreciated by those skilled in the art that computers or other electronic devices might utilize code realized in hardware to perform many or all the identified tasks without departing from the present invention. Those skilled in the art will understand that many variations on executable code may be found that implement exemplary methods within the spirit and the scope of the present invention.

The code or a copy of the code contained in computer program product 1460 may reside in one or more storage persistent media (not separately shown) communicatively coupled to computer system 1200 for loading and storage in persistent storage device 1270 and/or memory 1210 for execution by processor 1220. Computer system 1200 also includes I/O subsystem 1230 and peripheral devices 1240. I/O subsystem 1230, peripheral devices 1240, processor 1220, memory 1210, and persistent storage device 1270 are coupled via bus 1250. Like persistent storage device 1270 and any other persistent storage that might contain computer program product 1260, memory 1210 is a non-transitory media (even if implemented as a typical volatile computer memory device). Moreover, those skilled in the art will appreciate that in addition to storing computer program product 1260 for carrying out the processing described herein, memory 1210 and/or persistent storage device 1270 may be configured to store the various data elements referenced and illustrated herein.

Those skilled in the art will appreciate computer system 1200 illustrates just one example of a system in which a computer program product in accordance with an embodiment of the present invention may be implemented. To cite but one example of an alternative embodiment, storage and execution of instructions contained in a computer program product in accordance with an embodiment of the present invention may be distributed over multiple computers, such as, for example, over the computers of a distributed computing network.

1. A method of computerized processing of output of a quantitative polymerase chain reaction (qPCR) instrument, the qPCR instrument being configured to generate amplification data, referred to herein as amplification curves, an amplification curve comprising data corresponding to fluorescence measurements obtained for a plurality of thermal cycles of a qPCR instrument used to conduct a qPCR assay on a biological sample, the method implemented by executing, using one or more computer processors, processing comprising:
pre-processing a plurality of amplification curves generated by the qPCR instrument to obtain, for an amplification curve of the plurality of amplification curves, engineered features and a pre-processed amplification curve;
processing, by one or more amplification-calling deep learning networks, the pre-processed amplification curve and one or more of the engineered features to generate amplification call data;
processing, by one or more curve-quality calling deep learning networks, the pre-processed amplification curve to generate curve-quality call data; and
using the amplification call data and the curve quality call data to provide an interactive electronic graphical user interface (GUI) configured to facilitate user evaluation of the output of the qPCR instrument.

2. The method of claim 1 wherein using the amplification call data and the curve-quality data comprises determining whether the amplification call data should be invalidated.

3. The method of claim 2 wherein determining whether the amplification call data should be invalidated is based on one or more user settings in the GUI.

4. The method of claim 2 wherein determining whether the amplification call data should be invalidated is based on the curve-quality call data failing to meet pre-defined criteria.

5. The method of claim 4 wherein the pre-defined criteria is set by a user via the GUI.

6. The method of claim 1 wherein the amplification call data comprises class and/or class probability data corresponding to at least an amplified class and/or a non-amplified class.

7. The method of claim 1 wherein the amplification call data comprises call confidence data.

8. The method of claim 1 wherein the curve-quality call data comprises class and/or class probability data corresponding to at least a clean curve class and/or a problem curve class.

9. The method of claim 1 wherein the curve-quality call data comprises call confidence data.

10. The method of claim 1 wherein an amplification call generated by the one or more amplification-calling deep learning networks is compared with an amplification result generated by a pre-determined non-neural network based amplification-calling algorithm executed on the same pre-processed amplification curve to determine whether to evaluate the curve-quality call data for determining whether to invalidate the amplification call generated by the one or more amplification-calling deep learning networks.

11. The method of claim 10 further comprising evaluating curve-quality call data for a plurality of curves corresponding to results from a sample well to determine whether to invalidate all amplification calls associated the sample well.

12. The method of claim 10 wherein the non-neural network based amplification-calling algorithm comprises comparing a cycle threshold (Ct) value corresponding to the amplification curve with a Ct value assigned to the qPCR assay run on the qPCR instrument to generate the amplification curve.

13. The method of claim 10 wherein the non-neural network based amplification-calling algorithm comprises a Cycle Relative Threshold (Crt) algorithm.

14. The method of claim 1 wherein one or more of the engineered features corresponding to the application curve are processed by the one or more curve-quality calling deep learning networks along with the pre-processed amplification curve to generate the curve-quality call data.

15. The method of claim 1 wherein the engineered features comprise one or more features selected from a group consisting of: curve derivative features, time series features, PCRedux features, and Cycle Relative Threshold (Crt) algorithm features.

16. The method of claim 1 wherein the one or more amplification-calling deep learning networks comprise an ensemble of similarly structured but differently trained deep learning networks.

17. The method of claim 1 wherein the one or more curve-quality calling deep learning networks comprise an ensemble of similarly structured but differently trained deep learning networks.

18. A non-transitory computer readable medium storing instructions that, when executed by one or more computer processors, execute processing according to the method of claim 1.

19. A computer system comprising one or more processors configured to execute processing according to the method of claim 1.

20-84. (canceled)
